(19) United States
(12) Patent Application Publication
(10) Pub. No.: US 2007/0092463 A1
(43) Pub. Date: Apr. 26, 2007

(54) Semi (soft)-solid antiperspirant compositions

(76) Inventors: Joo W. Kim, Mississauga (CA); Mike Hooper, Mississauga (CA)

Correspondence Address: The Halvorson Law Firm, Ste 130, 1757 E. Baseline Rd., Gilbert, AZ, 85233, US

(21) Appl. No.: 11/259,172

(22) Filed: Oct. 26, 2005

(51) Int. Cl. A61K 8/28; A61K 8/26

(52) U.S. Cl. 424/66; 424/68

(57) ABSTRACT

Stable semi-solid anhydrous antiperspirant compositions comprising an antiperspirant powder, a volatile silicone and non-volatile materials, a primary gellant material comprising Glyceryl monostearate, secondary gellants, oil absorbents, and coupling agents. The resultant products exhibit minimal syneresis with any dispensing package and exhibiting dry feeling when applied to skin.

The present invention relates to anhydrous antiperspirant semi (soft) solid stick comprising a glycerin monostearate as main gelling agent, auxiliary gellants, a volatile silicone and non-volatile material, coupling agent, and oil absorbent material. This stick exhibit minimal syneresis with non-pressure relief dispensing package or pressure relief dispensing package and dry feeling when applied to skin.

### BACKGROUND AND PRIOR ART

Generally, antiperspirants are comprised of an antiperspirant active contained within a carrier. Hard carriers form stick type antiperspirants, which are applied directly by rubbing onto the coverage area. Liquid carriers form liquid type antiperspirants that are applied by structures such as “roll-on” applicators.

Lately there is great interest in antiperspirants that exhibit the benefits of both, the ease of application of a liquid and the dry-feel and direct application of a stick. These gelled or creamed antiperspirants are generally known as soft solids. These soft solid antiperspirants have harnesses between that of solid antiperspirants and liquid antiperspirants.

U.S. Pat. No. 4,526,780 prepared an anhydrous paste or cream type antiperspirant containing a bentonite gel as a suspending/thickening agent, fatty acid metal salts as a stability agent to prevent syneresis, talc as an oil absorbent material, antiperspirant actives, volatile silicone, and propylene carbonate as a wetting agent.

U.S. Pat. No. 4,937,069 prepared an anhydrous semi-solid antiperspirant containing a fumed silica as a thickening/suspending agent, organic waxes as thickening/solid emollient, volatile and non-volatile emollients, hydrophilic surfactants to reduce staining, and antiperspirant actives.

U.S. Pat. Nos. 5,718,890, 5,902,571, 5,871,717, 5,891,424, 5,902,570 disclose an anhydrous cream antiperspirant composition containing triglyceride gellants comprising glyceryl tribehenate and other triglycerides. This type of formula needs to use a special dispensing package that is described in U.S. Pat. No. 5,000,356.

U.S. Pat. No. 5,882,637 discloses an anhydrous cream antiperspirant composition containing fatty alcohols having from about 20 to about 60 carbon atoms.

U.S. Pat. No. 6,231,841 discloses an anhydrous solid or soft solid having at least one sterol and at least one sterol ester.

However, it has been found that these quasi-solid antiperspirants suffer from solvent syneresis, the weeping of solvent from the antiperspirant matrix either under pressure or during storage.

U.S. Pat. Nos. 5,000,356 and 4,865,231 disclose dispensing packages that automatically relieve residual pressure on soft solid antiperspirants and deodorants, preventing weeping onto the application surface of the dispenser.

These pressure relieving packages, while minimizing the problem of solvent syneresis, they do not address the root problem of syneresis. Therefore there is a current and ongoing need for soft solid antiperspirants that exhibit reduce syneresis while under the residual pressure exhibited after a soft solid antiperspirant has been dispensed.

### SUMMARY OF THE INVENTION

The anhydrous compositions of this invention are semi (soft)-solid type antiperspirant sticks that exhibit minimal syneresis.

The composition according to the present invention exhibits a viscosity ranging between in 5,000-2,000,000 cps as measured with Brookfield DV-II+ viscometer, Helipath T-bar Spindle # T-F @ 5 RPM, 23-26° C.

The composition according to the present invention comprises as essential constituents: an anhydrous liquid carrier, such as a volatile silicone solvent; a primary gellant: such as glycerin monostearate; a secondary gellant, such sorbitan monostearate and/or tribehenin and/or hydrogenated castor oil; and at least one antiperspirant active.

Optionally, the composition according to the present invention comprises: residue masking materials, such as dimethicone (5-60,000 cts), petrolatum, and/or other non-volatile emollients; coupling agent, such as glycol monooleate and/or Sorbitan tristearate; an oil absorbent, such as fumed silica and hydrophobically modified natural polymers; and fragrances.

The novel features that are considered characteristic of the invention are set forth with particularity in the appended claims. The invention itself, however, both as to its structure and its operation together with the additional object and advantages thereof will best be understood from the following description of the preferred embodiment of the present invention when read in conjunction with the accompanying drawings. Unless specifically noted, it is intended that the words and phrases in the specification and claims be given the ordinary and accustomed meaning to those of ordinary skill in the applicable art or arts. If any other meaning is intended, the specification will specifically state that a special meaning is being applied to a word or phrase. Likewise, the use of the words “function” or “means” in the Description of Preferred Embodiments is not intended to indicate a desire to invoke the special provision of 35U.S.C.§112, paragraph 6 to define the invention. To the contrary, if the provisions of 35U.S.C.§112, paragraph 6, are sought to be invoked to define the invention(s), the claims will specifically state the phrases “means for” or “step for” and a function, without also reciting in such phrases any structure, material, or act in support of the function. Even when the claims recite a “means for” or “step for” performing a function, if they also recite any structure, material or acts in support of that means of step, then the intention is not to invoke the provisions of 35U.S.C.§112, paragraph 6. Moreover, even if the provisions of 35U.S.C.§112, paragraph 6, are invoked to define the inventions, it is intended that the inventions not be limited only to the specific structure, material or acts that are described in the preferred embodiments, but in addition, include any and all structures, materials or acts that perform the claimed function, along with any and all known or later-developed equivalent structures, materials or acts for performing the claimed function.

### DESCRIPTION OF THE PREFERRED EMBODIMENT

The present invention is a soft solid antiperspirant composition that exhibit minimal syneresis. The composition according to the present invention comprises an anhydrous liquid carrier, a primary gellant a secondary gellant and an antiperspirant active.

Anhydrous Liquid Carrier (Volatile Silicones)

Cyclomethicones are employed in this invention as a main anhydrous liquid carrier. Cyclomethicones are volatile silicon oil that impart a smooth and silky and dry skin feel after topical application of these compositions onto the skin. To achieve the full advantage of the present invention, cyclomethicones that have average 3 to 5 membered ring structures are used. These volatile silicones are available commercially under the trade names SILICONE 244 FLUIDE, SILICONE 245 FLUIDE, SILICONE 344 FLUIDE, SILICONE 345 FLUIDE, and SILICONE 246 FLUIDE (from Dow Coming Co.), SF1202 (from General Electric), and SILICONE 7207 and 7158 (from Union Carbide co.). Volatile silicones are used in this invention in a proportion of up to 70%, preferably between 30-60%.

Primary Gellant

In order to minimize syneresis, a suitable gellant is critical to the present invention. To find out suitable gellants in compositions comprising 30-60% cyclomethicone, 5 parts of gellant was dissolved in 50 parts of cyclomethicone at 60-110° C. and cooled to 20-25° C., after which the gelling effect was tested. The results of these tests are summarized in Table 1 below,

TABLE 1
The gelling effect of single gellants

| | Solubility in | |
|---|---|---|
| | Cyclomethicone at | |
| Gellant | High Temperature | Gelling Effect after Cooling |
| Stearyl Alcohol | soluble | poor, hard, big plate type |
| (MP 58° C.) | | |
| Cetyl Alcohol | soluble | poor, hard, big plate type |
| (MP 49.3° C.) | | |
| Behenyl Alcohol | soluble | good, soft, fine needle, big |
| (MP 70.5° C.) | | plate after 2 days |
| Cetyl esters | soluble | poor, hard, big plate |
| Hydrogenated | insoluble | bad |
| Castor oil | | |
| (MP 80° C.) | | |
| Candelilla wax | insoluble | good, hard, needle to amorphous |
| (MP 68-72° C.) | (partially) | |
| Paraffin wax | soluble | good, hard, needle to amorphous |
| (MP 50-70° C.) | | |
| Microcrystalline | soluble | good, soft, very fine needle |
| wax | | |
| Ozokerite | soluble | good, soft, very fine amorphous |
| C18-36 Acid | insoluble | good, soft, needle to amorphous |
| Triglyceride | (partially) | |
| Tribehenin | soluble | good, soft, amorphous |
| C18-36 Acid | Insoluble | poor, hard, needle to amorphous |
| Glycol Ester | (partially) | |
| Glyceryl | soluble | good, soft, very fine amorphous |
| monostearate | | |
| (MP 55-58° C.) | | |
| Glycol | soluble | bad, big hexagonal plate |
| monostearate | | |
| Sorbitan | soluble | poor, amorphous |
| monostearate | | |
| Sorbitan | soluble | bad, very fine amorphous |
| tristearate | | |

It was found that fatty alcohols make hard structures, big plate type crystals, that are brittle. This type of gellant is not suitable for soft solid antiperspirants.

It was found that vegetable waxes make the structures that have very fine particles but usually include resin type substances that are not soluble in cyclomethicone at high temperature and after cooling, these resins become big wax particles that make the composition inhomogeneous.

It was found that tribehenin, which is one of synthetic waxes and are already described by U.S. Pat. Nos. 5,718,890, 5,902,571, 5,871,717, 5,891,424, 5,902,570, makes a soft structure that has fine amorphous particles and suitable for soft solid type stick. However, the gelling property was dependent upon cooling condition. Other synthetic waxes included resin type substances that render the composition inhomogeneous.

It was found that microcrystalline wax, and ozokerite make a good soft structure that has fine particles and are suitable for soft solid type antiperspirants. However, paraffin wax makes a hard and brittle structure that is not suitable for soft solid type antiperspirants.

It was found that glyceryl monostearate make a good soft structure that has fine particles and is less sensitive for cooling condition.

It was found that sorbitan monostearate has poor gelling property but make very fine amorphous type particles that is similar to tribehenin.

It was found that glycol monostearate and distearate make big hexagonal type crystals and also have poor gelling properties.

The gelling effects of mixutures of the gellants listed in table 1 are summarized in Table 2. To test these mixtures, 5-7 parts total of gellants were dissolved in 50 parts of cyclomethicone at 60-110° C. and cooled to 20-25° C. The results are summarized in Table 1 below.

TABLE 2
Gelling effect of the gellants mixture.

| | Solubility in | |
|---|---|---|
| | Cyclomethicone | |
| | at High | |
| Gellant Mixture | Temperature | Gelling Effect |
| Microcrystalline wax/ | soluble | poor, soft needle |
| Glyceryl monostearate (1:1) | | to plate |
| Ozokerite/ | soluble | good, soft fine |
| Glyceryl monostearate (1:1) | | amorphous |
| Sorbitan monostearate/ | soluble | good, soft fine |
| Glyceryl monostearate (1:1) | | amorphous |
| Sorbitan monostearate/ | soluble | good, soft fine |
| Glyceryl monostearate (1:6) | | amorphous |
| Sorbitan tristearate/ | soluble | good, soft fine needle |
| Glyceryl monostearate (1:1) | | to fine amorphous |
| Sorbitan tristearate/ | soluble | good, soft fine needle |
| Glyceryl monostearate (1:6) | | to fine amorphous |
| Tribehenin/ | soluble | poor, soft amorphous |
| Glyceryl monostearate (1:1) | | to needle |
| Sorbitan tristearate/ | soluble | good, soft, fine |
| Glyceryl monostearate (1:1) | | amorphous |
| Total 7 parts in 50 parts of | | |
| Cyclomethicone | | |
| Sorbitan tristearate/ | soluble | good, soft, fine |
| Glyceryl monostearate (1:6) | | amorphous |
| Total 7 parts in 50 parts of | | |
| Cyclomethicone | | |

It was surprising discovered that mixing glycerol monostearate with a secondary gellant synergistically creates a superior gellant that resists syneresis.

The effects of different masking materials that are employed in these compositions are summarized in Table 3.

TABLE 3
The effect of residue masking material.

| | Solubility in | |
|---|---|---|
| | Cyclomethicone at | |
| Gellant Mixture | High Temperature | Gelling Effect |
| Sorbitan monostearate | soluble | good, soft very fine |
| Glyceryl monostearate (1:1) | | amorphous |
| Total 7 parts to 50 | | |
| parts Cyclomethicone + | | |
| Petrolatum 5 Part | | |
| Sorbitan monostearate | not soluble | bad, big amorphous |
| Glyceryl monostearate (1:1) | | and not homogenous |
| Total 7 parts to 50 | | |
| parts Cyclomethicone | | |
| Dimethicone 5 Part | | |
| Sorbitan monostearate | soluble | good, soft very |
| Glyceryl monostearate (1:1) | | fine needle to plate |
| Total 7 parts to 50 | | |
| parts Cyclomethicone + | | |
| Mineral Oil 5 Part | | |
| Tribehenin Glyceryl | soluble | good, soft needle |
| monostearate (1:6) | | to plate |
| Total 7 parts to 50 | | |
| parts Cyclomethicone + | | |
| Petrolatum 5 Part | | |

Coupling Agent

The effect of non-ionic surfactant that are employed as coupling agent between hydrophilic active ingredient and lipophilic anhydrous carrier are summarized in Table 4.

TABLE 4
The effects of non-ionic surfactants.

| | Solubility in | |
|---|---|---|
| | Cyclomethicone at | |
| Gellant Mixture | High Temperature | Gelling Effect |
| Sorbitan monostearate | soluble | good, soft very fine |
| Glyceryl monostearate (1:1) | | amorphous |
| Total 7 parts to | | |
| 50 parts Cyclomethicone + | | |
| Petrolatum 5 Part | | |
| Sorbitan monooleate 2 part | | |
| Tribehenin Glyceryl | soluble | good, soft very fine |
| monostearate (1:6) | | amorphous |
| Total 7 parts to 50 | | |
| parts Cyclomethicone + | | |
| Petrolatum 5 Part | | |
| Sorbitan monooleate 2 part | | |

The non-ionic surfactants change the structure of gellant from crystal to very fine amorphous type particles and usually lowered the crystallization temperature.

Antiperspirant Materials

The present composition essentially contain powdered aluminum and/or zirconium salt that has an average particle size less than about 1000 microns, and preferably less than 100 microns, more preferably from about 1 to about 50 microns. Preferred salt include aluminum, zirconium and aluminum-zirconium halides and halohydrate salts such as aluminum chlorohydrates, aluminum zirconium tetra (or) tri chlorohydrate glycine (or propylene glycol) complex. The amount of antiperspirant material should be at least 5.0% and preferably 20.0-30.0%. Some activated salts are preferred to get enhanced antiperspirant activity.

A number of products of this salt are available commercially. These include such as Westchlor DM, ZR, and 200 series (from Westwood chemical corporation) and Rezal, Micro-Dry, Reach series (from Reheis Inc.).

Oil Absorbant and Suspending Agent

To absorb the excess amount of silicon oil, fumed silica, starch derivatives and (or) clay are employed in this invention. The fumed silica that employed in this invention should have an average particle size in the range of from about 0.001 micron to about 0.05 micron, and preferably in the range of from about 0.01 micron to about 0.02 micron. This suitable finely divided silica is inorganic oxide and available from Degussa (Aerosil 200) and Cabot (Cab-O-Sil). The amount of this silica in this composition is from about 1% to about 2%.

The clay that is employed in this invention is Bentone (N L Industry). This is a trade name of hydrophobic bentonites and hectorities. Specific examples of Bentones are Benton 34, and Bentone 27 and the amount in this composition is from about 0% to 3%.

The starch derivative that is employed in this invention is aluminum starch octenylsuccinate that is available commercially as Dry Flo PC or Pure (National Starch & Chemical). From around 1% to around 20% is employed in this composition.

The following specific examples are illustrative of the present invention. However, it should be understood that the present invention is not limited to the specific examples set forth below. All amounts of various ingredients are by weight unless otherwise specified.

### EXAMPLE 1

| Ingredient | % |
|---|---|
| Cyclomethicone pentamer | 48.00 |
| Glyceryl monostearate | 6.00 |
| Petrolatum | 5.00 |
| Glycerol monooleate | 2.00 |
| Tribehenin | 1.0 |
| Aluminum Zirconium trichlorohydrex glycine | 25.00 |
| Fumed Silica | 2.00 |
| Aluminum Starch Octenylsuccinate | 10.00 |
| Fragrance | 1.00 |

All of the listed components except fragrance are combined and heated to 70-72° C. with agitation. The mixture is cooled to about 60-63° C. and added fragrance and homogenized. Then the mixture is filled into jar and allowed to cool. The viscosity of this anhydrous cream should be in the range from about 700,000 cps to about 2,000,000 cps (Brookfield DV-II+ viscometer, Helipath T-bar Spindle # T-F @ 5 RPM, 23-26° C.).

### EXAMPLE 2

| Ingredient | % |
|---|---|
| Cyclomethicone pentamer | 49.00 |
| Glyceryl monostearate | 5.00 |
| Petrolatum | 5.00 |
| Glycerol monooleate | 2.00 |
| Hydrogenated Castor wax | 1.00 |
| Aluminum Zirconium trichlorohydrex glycine | 25.00 |
| Fumed Silica | 2.00 |
| Aluminum Starch Octenylsuccinate | 10.00 |
| Fragrance | 1.00 |

All of the listed components except Fragrance and Aluminum Starch Octenylsuccinate are Combined and heated to 82-85° C. with agitation. The mixture is cooled to about 78-80° C. and added Aluminum Starch Octenylsuccinate and cooled to about 64-67° C. and add fragrance and homogenized. Then the mixture is filled into jar and allowed to cool. The viscosity of this anhydrous cream should be in the range from about 700,000 cps to about 2,000,000 cps (Brookfield DV-II+ viscometer, Helipath T-bar Spindle # T-F @ 5 RPM, 23-26° C.).

### EXAMPLE 3

| Ingredient | % |
|---|---|
| Cyclomethicone pentamer | 43.90 |
| Glyceryl monostearate | 3.00 |
| Petrolatum | 5.00 |
| Glycerol monooleate | 2.00 |
| Sorbitan monostearate | 3.00 |
| Aluminum Zirconium trichlorohydrex glycine | 25.00 |
| Fumed Silica | 2.00 |
| Aluminum Starch Octenylsuccinate | 15.00 |
| Fragrance | 1.00 |

All of the listed components except Fragrance are Combined and heated to 70-72° C. with agitation. The mixture is cooled to about 60-63° C. and added fragrance and homogenized. And then the mixture is filled into jar and allowed to cool. The viscosity of this anhydrous cream should be in the range from about 700,000 cps to about 2,000,000 cps (Brookfield DV-II+ viscometer, Helipath T-bar Spindle # T-F@ 5 RPM, 23-26° C.).

### EXAMPLE 4

| Ingredient | % |
|---|---|
| Cyclomethicone Tetramer | 36.00 |
| Mineral Oil | 15.00 |
| Hydrogenated Castor wax | 6.00 |
| Tribehenin | 2.00 |
| Glycerol monooleate | 5.00 |
| Aluminum Zirconium trichlorohydrex glycine | 25.00 |
| Aluminum Starch Octenylsuccinate | 10.00 |
| Fragrance | 1.00 |

All of the listed components except Fragrance and Aluminum Starch Octenylsuccinate are Combined and heated to 82-85° C. with agitation. The mixture is cooled to about 78-80° C. and added Aluminum Starch Octenylsuccinate and cooled to about 70-72° C. and add fragrance and homogenized. Then the mixture is filled into jar and allowed to cool. The viscosity of this anhydrous cream should be in the range from about 700,000 cps to about 2,000,000 cps (Brookfield DV-II+ viscometer, Helipath T-bar Spindle# T-F @ 5 RPM, 23-26° C.).

### EXAMPLE 5

| Ingredient | % |
|---|---|
| Cyclomethicone pentamer | 52.00 |
| Glyceryl monostearate | 12.00 |
| Petrolatum | 3.00 |
| Dimethicone | 5.00 |
| Glycerol monooleate | 2.00 |
| Aluminum Zirconium trichlorohydrex glycine | 25.00 |
| Fragrance | 1.00 |

All of the listed components except Fragrance are Combined and heated to 73-75° C. with agitation. The mixture is cooled to about 57-61° C. and added fragrance and homogenized. And then the mixture is filled into jar and allowed to cool. The viscosity of this anhydrous cream should be in the range from about 700,000 cps to about 2,000,000 cps (Brookfield DV-II+ viscometer, Helipath T-bar Spindle # T-F @ 5 RPM, 23-26° C.).

### EXAMPLE 6

| Ingredient | % |
|---|---|
| Cyclomethicone pentamer | 48.00 |
| Glyceryl monostearate | 6.00 |
| Dimethicone | (60,000 cts) |
| Glycerol monooleate | 2.00 |
| Tribehenin | 1.00 |
| Aluminum Zirconium trichlorohydrex glycine | 25.00 |
| Aluminum Starch Octenylsuccinate | 10.00 |
| Fumed Silica | 2.00 |
| Fragrance | 1.00 |

All of the listed components except fragrance are combined and heated to 70-72° C. with agitation. The mixture is cooled to about 60-63° C. and added fragrance and homogenized. And then the mixture is filled into jar and allowed to cool. The viscosity of this anhydrous cream should be in the range from about 700,000 cps to about 2,000,000 cps (Brookfield DV-II+ viscometer, Helipath T-bar Spindle # T-F @ 5 RPM, 23-26° C.).

The example compositions above were tested to determine their resistance to syneresis by comparing these formulations with other commercially available soft solid formulations. While all formulations performed identically under storage in various different positions, upright, on their sides and inverted, only the compositions according to the present invention, the compositions containing glyceryl monostearate as a primary gellant and at least on secondary gellant, exhibited minimal syneresis under the pressure of a half turn of a non-reciprocating screw mechanism dispenser. The commercially available soft solid antiperspirants all exhibited significant syneresis and hardening of their compositions as a result of the syneresis. The composition according to the present invention only exhibited a mild condensation above the composition with no detectable discoloration or hardening of the composition under pressure.

The preferred embodiment of the invention is described above in the Drawings and Description of Preferred Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated.

What is claimed is:

1. An anhydrous semi-solid antiperspirant composition comprising an antiperspirant active, a liquid carrier and a primary gellant comprising glyceryl monostearate, at least one secondary gellant.

2. The composition according to claim 1 having a viscosity of from about 5,000 cps to about 2,000,000 cps.

3. The composition of claim 1 wherein said an antiperspirant active is selected from the group consisting of powdered aluminum and zirconium salts or complexes and mixtures thereof and is present in the composition in a weight percentage ranging between 5.0% -30.0%.

4. The composition of claim 1 wherein said a liquid carrier is a volatile cyclomethicone and contains 30.0% -60.0%.

5. The composition of claim 1 wherein said a gelling agent is a glyceryl monostearate and is present in the composition in a weight percentage ranging between 3.0-12.0%.

6. The composition of claim 1 wherein said at least one secondary gelling agents are hydrogenated castor oil and/or sorbitan monostearate and/or tribehenin and is present in the composition in a weight percentage ranging between 0.5 -3.0%.

7. The composition of claim 1 wherein said coupling agents are glycol monooleate and/or sorbitan tristearate and is present in the composition in a weight percentage ranging between 0.5-3.0%.

8. The composition of claim 1 wherein said oil absorbents are and/or fumed silica and/or starch derivative and/or clay and is present in the composition in a weight percentage ranging between 0.5-20.0%.

9. The composition of claim 1 wherein said composition comprises about 1.0-10.0 % of residue masking materials.
